(19) United States
(12) Patent Application Publication
(10) Pub. No.: US 2016/0183921 A1
(43) Pub. Date: Jun. 30, 2016

(54) Monitor with Ultrasonic Scanning and Monitoring Functions, Ultrasonic Apparatus, and Corresponding Method

(71) Applicant: SHENZHEN MINDRAY BIO-MEDICAL ELECTRONICS CO., LTD., Shenzhen (CN)

(72) Inventors: Yong Li, Shenzhen (CN); Shuo Liu, Shenzhen (CN); Jin Liu, Shenzhen (CN); Haibo Song, Shenzhen (CN)

(21) Appl. No.: 14/860,500

(22) Filed: Sep. 21, 2015

Related U.S. Application Data
(63) Continuation of application No. PCT/CN2013/083100, filed on Sep. 9, 2013.

(30) Foreign Application Priority Data
Mar. 19, 2013 (CN) 201310087441.5

(51) Int. Cl. A61B 8/00; A61B 8/08; A61B 8/14

(52) U.S. Cl. CPC A61B 8/463; A61B 8/14; A61B 8/5223; A61B 8/483; A61B 8/486; A61B 8/488; A61B 8/485

(57) ABSTRACT

Disclosed is a monitor with ultrasonic scanning and monitoring functions including a monitoring function module (10), a monitoring sensor (20), an ultrasonic function module (12), an ultrasonic sensor (22) and a display apparatus (3). The monitoring function module (10) is used for mentoring a common physiological parameter. The ultrasonic function module (12) is used for monitoring a detected person by ultrasonically scanning to obtain a corresponding ultrasonic image and a physiological parameter thereof. The monitoring sensor (20) is coupled to the monitoring function module (10). The ultrasonic sensor (22) is coupled to the ultrasonic function module. The display apparatus (3) is coupled to the monitoring function module and the ultrasonic function module, and receives and displays the common physiological parameter as well as the ultrasonic image and the physiological parameter thereof. Also disclosed is a corresponding monitoring method. By means of the present disclosure, during monitoring the common physiological parameter, the ultrasonic image and the physiological parameter thereof can be obtained and displayed by performing ultrasonic scanning with a predetermined time period and duration, thereby better meeting clinical requirements.

### FIELD OF THE INVENTION

The present disclosure relates to medical devices, especially to monitors with ultrasonic scanning and monitoring functions, ultrasonic apparatuses, and corresponding methods.

### BACKGROUND OF THE INVENTION

Monitors are often used to monitor patients' vital signs when diagnosing and clinically monitoring critical patients. Existing monitors provides cardiac monitoring information which contains parameters about electrocardiograph monitoring (ECG or EKG), blood pressure, oxygen saturation (SPO2) and etc.. The EKG parameter is susceptible to interference such as poor electrode contact and improper manual operation, which may cause the EKG parameter to deviate from actual situation. Accordingly, false alarms may lead to alarm fatigue, resulting in affecting doctors to deal with emergency situation in a timely manner. On the other hand, the blood pressure parameter shown in a display interface of the monitor is usually a numeric-type parameter which has disadvantages of discontinuity and indirect representation of cardiac state. For example, when a low blood pressure is shown in the display interface, a doctor could not tell that is it a problem of heart failure or insufficient blood volume. While with regard to the SPO2 parameter, there may be defects of instability and no real time with the existing monitors.

In some clinical situations, such as heart monitoring in a non-cardiac surgery, observing patient transfer, and supervising intensive care unit (ICU), it may take a long time to monitor real-time parameters of heart movement, for example, noninvasively monitoring hemodynamic parameters such as ejection fraction and left ventricular fraction shortening. Such parameters can only be obtained by not a general monitor but an ultrasonic diagnostic system.

However, a traditional ultrasonic diagnostic system is only disposed in ultrasonic department. At there, a doctor needs to hold an ultrasonic probe for scanning a part to be examined of a patient, thereby obtaining ultrasonic images for a short time diagnosis. With regard to the above-mentioned clinical situations, i.e. monitoring the patient's physiological parameters (including real-time information of organ tissues, such as hemodynamic parameters) for a long time, the existing ultrasonic diagnostic systems could be unable to be applied in such a long time monitoring and hardly meet a wider range of clinical needs. In addition, in the prior art, the probe used for ultrasonic scanning cardiac hemodynamic is usually a probe placed in the esophagus. The patient may feel great discomfort under a long time of monitoring with the esophagus probe.

### SUMMARY OF THE INVENTION

In order to eliminate one of the above drawbacks of the prior art, monitors having ultrasonic scanning function, ultrasonic apparatuses and corresponding methods are proposed in the present disclosure. With integrating functions of monitors and ultrasonic scanning systems, they can perform ultrasonic scanning periodically on a patient, display obtained ultrasonic images and parameters on the monitor, and bring no discomfort to the patient.

To solve the above-mentioned technical problems, according to an embodiment of the present disclosure, a monitor with ultrasonic scanning and monitoring functions is provided, including a monitoring function module, an ultrasonic function module, a monitoring sensor, an ultrasonic sensor and a display apparatus.

The monitoring function module may be used for monitoring a common physiological parameter.

The ultrasonic function module may be used for monitoring a tagged object by ultrasonically scanning the tagged object with a predetermined time interval to obtain and process echo signals from the tagged object.

The monitoring sensor may be coupled to the monitoring function module, and the monitoring function module may be coupled to the ultrasonic function module.

The display apparatus may be coupled to the monitoring function module and the ultrasonic function module, and may receive and display the common physiological parameter derived from the monitoring function module and the processed echo signals derived from the ultrasonic function module.

Furthermore, the ultrasonic sensor may be attached directly and tightly on a fixed position of the tagged object's body surface, so as to scan a predetermined slice plane of the tagged object.

Furthermore, the monitor may also include a scanning period setting unit for setting the predetermined time interval and/or a duration of each scanning.

Furthermore, the monitor may also include:
a storage unit (16) for storing the common physiological parameter detected by the monitoring function module and/or the processed echo signals obtained by the ultrasonic function module within a predetermined time.

Furthermore, the process performed on the echo signal of the tagged object by the ultrasonic function module may include: performing digital process on the echo signals to acquire a digital-processing-step signal, and calculating and obtaining a numeric-type parameter, a waveform or a trend chart based on the digital-processing-step signal, wherein the digital-processing-step signal may be one of a radio frequency signal, a baseband signal, and an envelope signal.

Furthermore, the process performed on the echo signal of the tagged object by the ultrasonic function module may include: performing digital process on the echo signals to acquire a digital-processing-step signal, automatically calculating and analyzing the digital-processing-step signal or an ultrasonic image acquired by an imaging mode supported by the ultrasonic function module, and obtaining a numeric-type parameter, a waveform or a trend chart, wherein the digital-processing-step signal may be one of the following signals: a radio frequency signal, a baseband signal, and an envelope signal; and the imaging mode supported by the ultrasonic function module may be at least one of the following modes: B-mode imaging mode, M-mode imaging mode, color Doppler imaging mode, pulse wave imaging mode, elasticity imaging mode, 3D imaging mode and 4D imaging mode.

Furthermore, the display apparatus may be further provided with:
a common monitoring information display unit for displaying the common physiological parameter monitored by the monitoring function module; and
an ultrasonic information display unit for displaying the processed echo signals obtained by the ultrasonic function module.

Furthermore, the ultrasonic sensor may be a plurality of ultrasonic transducers connected to the ultrasonic function module, each ultrasonic transducer may be attached the tagged object's different positions of body surface, and the plurality of ultrasonic transducers may scan the tagged object synchronously and in real time.

Furthermore, the common physiological parameter monitored by the monitoring function module may include at least one of electrocardiogram monitoring parameter, blood pressure, oxygen saturation parameter and respiration parameter; and the ultrasonic function module may obtain at least one of cardiac ejection fraction, left ventricular fractional shortening, stroke volume, cardiac output, cardiac index, left ventricular end-diastolic volume and left ventricular end-systolic volume after processing the echo signals of the tagged object.

Furthermore, the monitoring function module and the ultrasonic function module may be independent of each other.

Furthermore, the monitoring function module and the ultrasonic function module may be connected directly or connected via a host of the monitor.

Furthermore, the monitoring function module and the ultrasonic function module may be detachable modules independent of each other, each pluggable connecting with a host of the monitor; and the host may provide corresponding pluggable interface for each module.

Accordingly, a monitoring method, which is implemented in the above-mentioned monitor and used for synchronously monitoring a common physiological parameter and ultrasonic scanning, is provided according to another aspect of an embodiment of the present disclosure, wherein the method may include the following steps:
monitoring a common physiological parameter of a tagged object through the monitoring function module, and displaying the monitored common physiological parameter on the display apparatus; and
performing ultrasonic scanning on human tissue through the ultrasonic function module, obtaining echo signals of the tagged object, processing the echo signals to form an image, a waveform or a numeric-type parameter and display on the display apparatus.

The step of performing ultrasonic scanning on human tissue through the ultrasonic function module to obtain echo signals of the tagged object may include:
allowing at least one ultrasonic probe to be directly attached tightly on a correspondingly fixed position of the tagged object's body surface, scanning a predetermined slice plane at each corresponding position of the tagged object to obtain echo signals from the corresponding position.

Furthermore, the step of processing the echo signals to form an image, a waveform or a numeric-type parameter and display on the display apparatus may include:
performing digital process on the echo signals to acquire a digital-processing-step signal, and calculating and obtaining a numeric-type parameter, a waveform or a trend chart based on the digital-processing-step signal.

Furthermore, the digital-processing-step signal may be one of a radio frequency signal, a baseband signal, and an envelope signal.

Furthermore, the step of processing the echo signals to form an image, a waveform or a numeric-type parameter and display on the display apparatus may include:
performing digital process on the echo signals to acquire a digital-processing-step signal, automatically calculating and analyzing the digital-processing-step signal or an ultrasonic image acquired by an imaging mode supported by the ultrasonic function module, and obtaining a numeric-type parameter, a waveform or a trend chart, wherein the digital-processing-step signal may be one of the following signals: a radio frequency signal, a baseband signal, and an envelope signal; and the imaging mode supported by the ultrasonic function module may be at least one of the following modes: B-mode imaging mode, M-mode imaging mode, color Doppler imaging mode, pulse wave imaging mode, elasticity imaging mode, 3D imaging mode and 4D imaging mode.

Accordingly, an ultrasonic apparatus for monitoring is provided according to another aspect of an embodiment of the present disclosure. The ultrasonic apparatus may include an ultrasonic function module, an ultrasonic sensor and a display apparatus, wherein:
the ultrasonic sensor may be coupled to the ultrasonic function module and scan a predetermined slice plane of a tagged object to obtain echo signals of the tagged object;
the ultrasonic function module may monitor the tagged object by scanning the tagged object with a predetermined time interval to obtain and process the echo signals of the tagged object acquired from the ultrasonic sensor; and
the display apparatus may be coupled to the ultrasonic function module, receive and display the processed echo signals derived from the ultrasonic function module.

Furthermore, the ultrasonic sensor may be attached tightly on a fixed position of the tagged object's body surface, so as to scan the predetermined slice plane of the tagged object.

Furthermore, the ultrasonic function module may further include:
a scanning period setting unit for setting the predetermined time interval and/or a duration of each scanning.

Furthermore, the ultrasonic function module may further include:
a storage unit for storing the processed echo signals obtained by the ultrasonic function module within a predetermined time.

Furthermore, the process performed on the echo signals of the tagged object by the ultrasonic function module may include: calculating the echo signals to obtain a numeric-type parameter, a waveform or a trend chart.

Furthermore, the process performed on the echo signals of the tagged object by the ultrasonic function module may include: performing digital process on the echo signals to acquire a digital-processing-step signal, automatically calculating and analyzing the digital-processing-step signal or an ultrasonic image acquired by an imaging mode supported by the ultrasonic function module, and obtaining a numeric-type parameter, a waveform or a trend chart, wherein the digital-processing-step signal may be one of the following signals: a radio frequency signal, a baseband signal, and an envelope signal; and the imaging mode supported by the ultrasonic function module may be at least one of the following modes: B-mode imaging mode, M-mode imaging mode, color Doppler imaging mode, pulse wave imaging mode, elasticity imaging mode, 3D imaging mode and 4D imaging mode.

Furthermore, the display apparatus may further be provided with:
a monitoring information display unit for displaying the common physiological parameter monitored by the monitoring function module; and
an ultrasonic image display unit for displaying the processed echo signals obtained by the ultrasonic function module.

Furthermore, the ultrasonic sensor may be a plurality of ultrasonic transducers connected to the ultrasonic function module, each ultrasonic transducer may be attached on the tagged object's different positions of body surface, wherein the plurality of ultrasonic transducers scan the tagged object synchronously and in real time.

Furthermore, the ultrasonic function module may obtain at least one of cardiac ejection fraction, left ventricular fractional shortening, stroke volume, cardiac output, cardiac index, left ventricular end-diastolic volume and left ventricular end-systolic volume after processing the echo signals of the tagged object.

Furthermore, the display apparatus may be provided with a display screen having at least one window for displaying the numeric-type parameter, the waveform or the trend chart by default according to the monitoring information display unit; and the display screen may be provided with a window which is triggered to display the ultrasonic image according to the monitoring information display unit.

Furthermore, the window displaying the ultrasonic image may be independent of the window displaying the numeric-type parameter, the waveform or the trend chart.

Furthermore, the window for displaying the ultrasonic image is or one of the window displaying the numeric-type parameter, the waveform or the trend chart.

The monitor with ultrasonic scanning and monitoring functions, ultrasonic apparatus and corresponding method provided in an embodiment of the present disclosure have the following advantages:
in an embodiment of the present disclosure, by means of integrating the functions of ultrasonic scanning and monitoring into a conventional monitor, and performing ultrasonic scanning with a predetermined time period and duration, the ultrasonic image and the physiological parameter can be obtained and displayed on the monitor, thereby better meeting clinical requirements;
the ultrasonic sensor adopted in the embodiment of the present disclosure can be attached on a patient's body surface for a long time, thereby ensuring the same predetermined slice plane for each scanning and improving accuracy of the acquired ultrasonic image, resulting in avoiding the risk of acoustical power caused by continuous scanning as well as the discomfort brought by the transesophageal probe in the prior art;

Moreover, in the embodiment of the present disclosure, the ultrasonic function module can be connected with a plurality of ultrasonic transducer, so that the plurality of ultrasonic transducers may scan the tagged object synchronously and in real time, which is convenient for ultrasonic scanning and monitoring multiple parts of the detected patient at the same time.

### BRIEF DESCRIPTION OF THE DRAWINGS

In order to more clearly illustrate the embodiments of the present disclosure or the technical solutions in the prior art, the accompanying drawings used in the embodiments or the prior art will be briefly introduced below. Apparently, the accompanying drawings described below are only some embodiments of the present disclosure; other drawings can also be obtained based on these accompanying drawings without creative work by those skilled in the art.

FIG. 1 is a schematic diagram of the structure of a monitor with ultrasonic scanning and monitoring functions according to an embodiment of the present disclosure;

FIG. 2 is a schematic diagram showing periodical scanning carried out in the ultrasonic function module shown in FIG. 1;

FIG. 3 is a schematic diagram of the structure of an ultrasonic apparatus for monitoring according to an embodiment of the present disclosure.

### DETAILED DESCRIPTION OF THE INVENTION

Taken in conjunction with the attached drawing of the present disclosure, the technical solution of the embodiments of the present disclosure will be clearly and completely described below. Obviously, the described embodiments are not all but merely some embodiments of the present disclosure. Based on the embodiments described herein, for those skilled in the art, other embodiments acquired without creative work belong to the scope of the protection of the present disclosure.

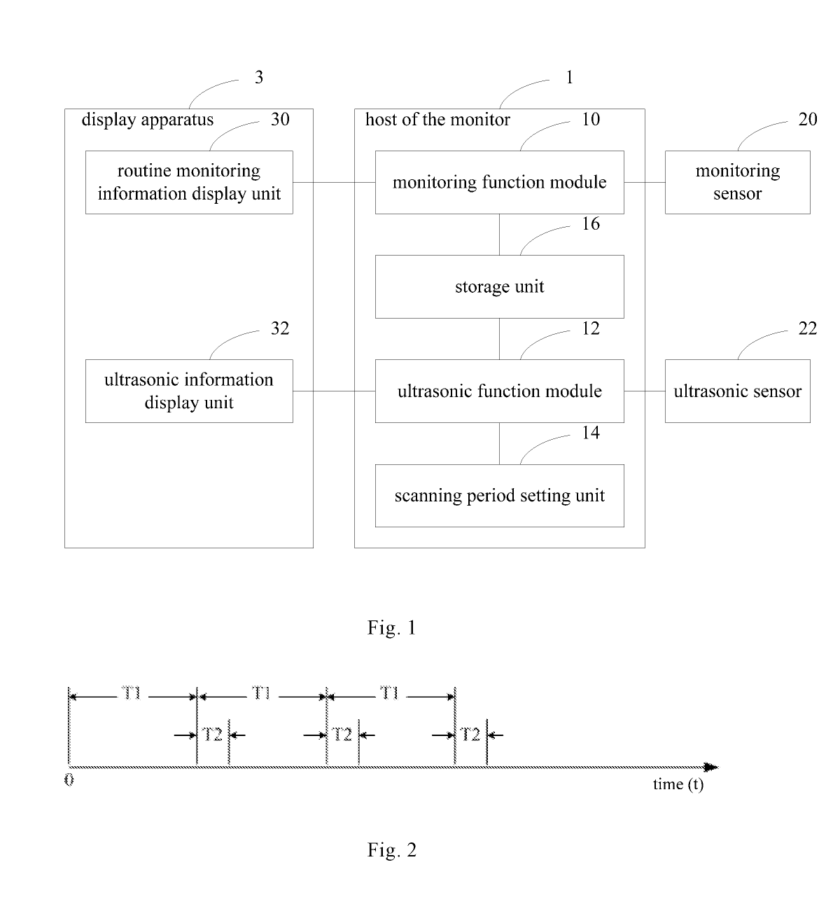

FIG. 1 is a schematic diagram of the structure of a monitor with ultrasonic scanning and monitoring functions in an embodiment of the present disclosure. The monitor with ultrasonic scanning and monitoring functions may include a host 1 of the monitor having a monitoring function module 10 and an ultrasonic function module 12, a monitoring sensor 20, an ultrasonic sensor 22 and a display apparatus 3.

The monitoring function module 10 can be used for monitoring a common physiological parameter (routine or conventional physiological parameter) which includes such as ECG parameter (electrocardiogram monitoring parameter), blood pressure, oxygen saturation parameter and respiration parameter. To be specific, the monitoring function module may be a function module integrating with monitoring at least one kind of parameters mentioned above, for example, a module integrated with functions of monitoring ECG, blood pressure, oxygen saturation and respiration parameter. The ECG parameter may further include, such as, heart rate, ventricular premature beat and etc..

The ultrasonic function module 12 may be used for monitoring a tagged object by ultrasonically scanning with the tagged object with a predetermined time interval to obtain and process echo signals from the tagged object. After processing the echo signals of the tagged object, the ultrasonic function module may obtain, such as, hemodynamic parameter and cardiac parameter. In an embodiment, such parameter may be at least one selected from a group consisting of cardiac ejection fraction, left ventricular fractional shortening, stroke volume, cardiac output, cardiac index, left ventricular end-diastolic volume and left ventricular end-systolic volume. The process performed on the echo signals of the detected by the ultrasonic function module 12 may include: calculating the echo signals to obtain a numeric-type parameter, a waveform or a trend chart. The numeric-type parameter may be physiological parameter information including hemodynamic parameter and cardiac parameter. In another embodiment, The process of the echo signals of the tagged object carried out by the ultrasonic function module 12 may include: generating a digital-processing-step signal by performing digital process on the echo signals, then optionally further generating an ultrasonic image with a selected or supported imaging mode, then automatically calculating and analyzing the digital-processing-step signal or an ultrasonic image acquired by an imaging mode supported by the ultrasonic function module to obtain a numeric-type parameter, a waveform or a trend chart. The digital-processing-step signal may be at least one selected from a group consisting of a radio frequency signal, a baseband signal, and an envelope signal. The imaging mode supported by the ultrasonic function module may be at least one imaging mode selected from a group consisting of B-mode (Brightness mode) imaging mode, M-mode (Motion-mode) imaging mode, 1D-space multi-point motion sequence diagram) imaging mode, color Doppler imaging mode, pulse wave (PW) imaging mode, elasticity imaging mode, 3D (three-dimension) imaging mode and 4D (four-dimension) imaging mode.

The monitoring sensor 20 may be coupled to the monitoring function module 10, and the monitoring function module 10 may be coupled to the ultrasonic function module 12.

The display apparatus 3, coupled to the host 1 of the monitor, may receive and display the common physiological parameter derived from the monitoring function module 10 and the processed echo signals derived from the ultrasonic function module 12.

In an embodiment, the display apparatus may be further provided with:
a common monitoring information display unit 30 for displaying the common physiological parameter monitored by the monitoring function module 10; and
an ultrasonic information display unit 32 for displaying the processed echo signals obtained by the ultrasonic function module 12.

In a specific implementation, different display windows may be arranged on the display screen of the display apparatus 3 for respectively displaying the common physiological parameter and the processed echo signals (image, waveform or numeric-type parameter).

Furthermore, the host 1 of the monitor may also include:
a storage unit 16 for storing the common physiological parameter detected by the monitoring function module 10 and/or the processed echo signals obtained by the ultrasonic function module 12 within a predetermined time. The stored common physiological parameter and the processed echo signals mentioned above can be replayed through the different display windows of the display apparatus 3 for providing reference for clinical diagnosis.

The ultrasonic sensor 22 can be attached tightly on the body surface of the tagged object (a patient). It could be attached on a fixed position of the body surface of the tagged object (such as a patient) for a long time, thereby ensuring the same slice plane for each scanning and improving accuracy of the acquired ultrasonic image.

Moreover, in an embodiment, the ultrasonic function module 12 can be connected with a plurality of ultrasonic transducers 22. Each ultrasonic transducer 22 can be attached tightly on the different positions of the tagged object's body surface, which is convenient for ultrasonic scanning and monitoring multiple parts of the detected patient at the same time.

In the above-mentioned embodiments, the ultrasonic function module 12 and the monitoring function module 10 can be integrated as a whole, such as integrated into the host 1 of the monitor. It can be understood that, in another embodiment, the ultrasonic function module 12 and the monitoring function module 10 are independent of each other. For example, the ultrasonic function module 12 and the monitoring function module 10 may both fixedly connected to a host of the monitor; or the ultrasonic function module 12 and the monitoring function module 10 may be detachable modules independent of each other, each pluggable connecting with a host of the monitor providing a corresponding pluggable interface reserved for each module. When one or some measuring modules are needed to be used, it can be done by simply plugging the corresponding measuring module to the host monitor.

It can be understood that, in other embodiments, the ultrasonic function module 12 could work independent of the monitoring function module 10, for example, the ultrasonic function module 12 could be used alone as a portable ultrasonic device.

In some embodiments, a time period to start the ultrasonic function module 12 and the duration for scanning can be set. To be specific, the host 1 of the monitor may include:
a scanning period setting unit 14 for setting the predetermined time interval and/or a duration of each scanning. Of course, the scanning period setting unit 14 may also be used only for setting a time interval for starting ultrasonic scanning or a duration of each scanning. When setting the above-mentioned time parameter related to scanning, the desired parameter can be inputted directly through a touch screen arrangement or a keyboard, a main control panel of the display apparatus. An alternative way to set the parameter is to offer a plurality of prepared mating parameters stored in the system for a user to choose. Each mating parameter of scanning time (including time for each scanning and interval of scanning) may be further corresponded to a default scanning time parameter under a certain application. For example, various types of prepared scanning time parameters can be predetermined based on different monitoring objects, consequently, a doctor selecting different monitoring objects means that different scanning time parameters may correspondingly be chosen. On the other hand, the scanning period mentioned here may be appreciated to be a fixed interval of time, a fixed scanning time, or an interval of time and scanning time which are varied based on a certain rule.

Referring to FIG. 2, it schematically illustrates periodical scanning carried out in the ultrasonic function module shown in FIG. 1. As seen from FIG. 2, the ultrasonic function module 12 may be starts periodically (every time interval T1) with a duration T2 for each scanning. The user (e.g. a doctor or a nurse) can use the above-mentioned scanning period setting unit 14 to set the values of T1 and T2 under B/M mode. For example, in a certain application, the user can set T1 to be 30 minutes and T2 to be 30 seconds, then the ultrasonic function module 12 will start scanning every 30 minutes with a duration of 30 second per scanning, and the result of each scanning can be stored in the storage unit 16.

Accordingly, a monitoring method, implemented in the above-mentioned monitor and explained by taken in conjunction with FIG. 1 and FIG. 2, is provided according to an embodiment of the present disclosure for synchronously monitoring a common physiological parameter and ultrasonic scanning. The method may include the following steps:
monitoring a common physiological parameter of a tagged object through the monitoring function module 10, and displaying the monitored common physiological parameter on the display apparatus; and
performing ultrasonic scanning on human tissue through the ultrasonic function module 12 to obtain echo signals of the tagged object, processing the echo signals to form an image, a waveform or a numeric-type parameter and display on the display apparatus 3.

The step of performing ultrasonic scanning on human tissue through the ultrasonic function module to obtain echo signals of the tagged object may include:
allowing at least one ultrasonic probe to be directly attached tightly on a correspondingly fixed position of the tagged object's body surface, scanning a predetermined slice plane at each correspondingly fixed position of the tagged object to obtain echo signals from the corresponding position.

The step of processing the echo signals to form an image, a waveform or a numeric-type parameter and display on the display apparatus may include:
directly computing the echo signals to obtain a numeric-type parameter, a waveform or a trend chart and display them on the display apparatus.

Or in another embodiment, the step of processing the echo signals to form an image, a waveform or a numeric-type parameter and display them on the display apparatus may include:
with the ultrasonic function module, performing digital process on the echo signals to generate a digital-processing-step signal, then optionally further generating an ultrasonic image with a selected or supported imaging mode, automatically calculating and analyzing the digital-processing-step signal or an ultrasonic image acquired by an imaging mode supported by the ultrasonic function module, and obtaining a numeric-type parameter, a waveform or a trend chart. The digital-processing-step signal may be at least one selected from a group consisting of a radio frequency signal, a baseband signal, and an envelope signal. The imaging mode supported by the ultrasonic function module may be at least one imaging mode selected from a group consisting of B-mode imaging mode, M-mode imaging mode, color Doppler imaging mode, pulse wave imaging mode, elasticity imaging mode, 3D imaging mode and 4D imaging mode.

A more specific detail can refer to the explanation of FIG. 1 and FIG. 2, which will not be repeated herein.

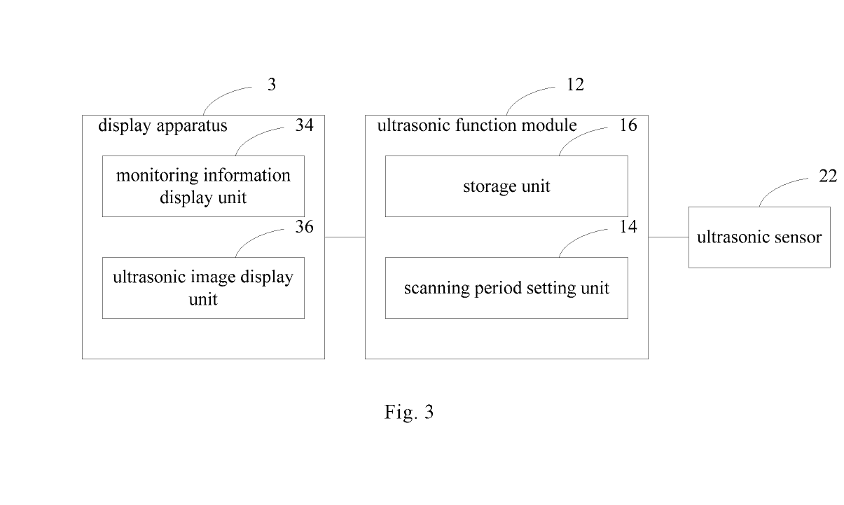

FIG. 3 is a schematic diagram of the structure of an ultrasonic apparatus for monitoring in an embodiment of the present disclosure. As seen from FIG. 3, the ultrasonic apparatus may include an ultrasonic function module 12, an ultrasonic sensor 22 and a display apparatus 3.

The ultrasonic sensor 22 may be coupled to the ultrasonic function module 12 for scanning a predetermined slice plane of a tagged object to obtain echo signals of the tagged object; specifically, the ultrasonic sensor may be attached tightly on a fixed position of the tagged object's body surface, so as to scan the same slice plane at the fixed position of the tagged object. The ultrasonic function module 12 may obtain at least one selected from a group consisting of cardiac ejection fraction, left ventricular fractional shortening, stroke volume, cardiac output, cardiac index, left ventricular end-diastolic volume and left ventricular end-systolic volume after processing the echo signals of the tagged object.

The ultrasonic function module 12 may be used for monitoring the tagged object by scanning the tagged object with a predetermined time interval, and obtaining and processing the echo signal of the tagged object acquired from the ultrasonic sensor 22.

The display apparatus 3, coupled to the ultrasonic function module 12, may receive and display the processed echo signals derived from the ultrasonic function module 12.

The ultrasonic function module 12 may further include:
a scanning period setting unit 14 used for setting the determined time interval for ultrasonic scanning started by the ultrasonic function module 12 and/or a duration of each scanning, wherein a detailed setting can refer to the above-mentioned description of FIGS. 2; and
a storage unit 16 used for storing the processed echo signals obtained by the ultrasonic function module 12 within a predetermined time.

The process performed on the echo signals of the tagged object by the ultrasonic function module 12 may include: calculating the echo signals to obtain a numeric-type parameter, a waveform or a trend chart.

Or in another embodiment, the process performed on the echo signal of the tagged object by the ultrasonic function module 12 may include: performing digital process on the echo signals to obtain a digital-processing-step signal, and calculating the digital-processing-step signal to obtain parameter formed of numeric type, waveform or trend chart for the purpose of monitoring; or obtaining an ultrasonic image according to the digital-processing-step signal and a selected imaging mode, and calculating based on the ultrasonic image to obtain a numeric-type parameter, a waveform or a trend chart. The imaging mode supported by the ultrasonic function module 12 may be at least one imaging mode selected from a group consisting of B-mode imaging mode, M-mode imaging mode, color Doppler imaging mode, pulse wave imaging mode, elasticity imaging mode, 3D imaging mode and 4D imaging mode.

The display apparatus 3 may further be provided with:
a monitoring information display unit 34 used for displaying the numeric-type parameter, the waveform or the trend chart acquired by the monitoring function module 12 according to the echo signals; and
an ultrasonic image display unit 36 used for displaying the processed echo signals obtained by the ultrasonic function module 12.

In a specific embodiment, the display apparatus 3 may be provided with a display screen having at least one window for displaying the numeric-type parameter, the waveform or the trend chart by default according to the monitoring information display unit 34; and the display screen may be provided with a window which is triggered to display the ultrasonic image according to the monitoring information display unit 36.

The window displaying the ultrasonic image may be independent of the window displaying the numeric-type parameter, the waveform or the trend chart.

Or the window displaying the ultrasonic image is or one of the window displaying the numeric-type parameter, the waveform or the trend chart, through which, the display window can be reused for displaying the ultrasonic image as well as the numeric-type parameter, the waveform or the trend chart respectively in different time periods.

“Triggered” can be implemented by using operational keys or options, that is, displaying the ultrasonic image on the display screen of the display apparatus through triggering an optional key or selecting a corresponding option for the sake of viewing by a doctor.

In other embodiments, the ultrasonic sensor 22 may be a plurality of ultrasonic transducers connected to the ultrasonic function module 12. Each ultrasonic probe 22 can be attached tightly on the tagged object's different positions of body surface. The plurality of ultrasonic transducers may scan the tagged object synchronously and in real time.

In the embodiments of the present disclosure, by means of integrating the functions of ultrasonic scanning and monitoring into a conventional monitor, and performing ultrasonic scanning with a predetermined time period and duration, the ultrasonic image and the physiological parameter can be obtained and displayed on the monitor, thereby better meeting clinical requirements;

The ultrasonic sensor adopted in the embodiment of the present disclosure can be attached on a patient's body surface for a long time, thereby ensuring the same slice plane for each scanning and improving accuracy of the acquired ultrasonic image, resulting in avoiding the risk of acoustical power caused by continuous scanning as well as the discomfort brought by the transesophageal probe in the prior art;

Moreover, in the embodiments of the present disclosure, the ultrasonic function module can be connected with a plurality of ultrasonic transducer, so that the plurality of ultrasonic transducers may scan synchronously and in real time scanning, which is convenient for ultrasonic scanning and monitoring multiple parts of the detected patient at the same time.

Those skilled in the art may understand that, all or part of the processes in the above-described embodiments of the method can be implemented by instructing relevant hardware through a computer program stored in a computer readable storage medium. When being executed, the program may include processes such as the above-described embodiments of the method. The medium may be a magnetic disk, an optical disk, a read-only storage memory (Read-Only Memory, ROM) or random access memory (Random Access Memory, RAM) and the like. “Coupled” referred to herein may include various contact and non-contact connection which is able to transmit a signal/power. Although a host of the monitor is defined herein, it can be appreciated that, the similar purpose can be achieved by an ultrasonic host and a monitoring function module integrated thereto, or by other medical devices or systems integrating with the ultrasonic function module and the monitoring function module, such as CT or MRI integrating with the ultrasonic function module and the monitoring function module.

The above reveal is intended to be preferred embodiments of the present disclosure only, and of course not to limit the scope of the disclosure as claimed, therefore equivalent changes made under this claimed disclosure are still within the scope of the disclosure.

What is claimed is:

1. A monitor with ultrasonic scanning and monitoring functions comprising a monitoring function module (10), an ultrasonic function module (12), a monitoring sensor (20), an ultrasonic sensor (22) and a display apparatus (3), wherein
the monitoring function module (10) monitors a common physiological parameter;
the ultrasonic function module (12) monitors a tagged object by scanning the tagged object with a predetermined time interval to obtain and process echo signals from the tagged object;
the monitoring sensor (20) is coupled to the monitoring function module, and the monitoring function module is coupled to the ultrasonic function module; and
the display apparatus (3) is coupled to the monitoring function module and the ultrasonic function module, receives and displays the common physiological parameter derived from the monitoring function module and the processed echo signals derived from the ultrasonic function module.

2. The monitor with ultrasonic scanning and monitoring functions according to claim 1, wherein the ultrasonic sensor (22) is attached tightly on a fixed position of the tagged object's body surface, so as to scan a predetermined slice plane of the tagged object.

3. The monitor with ultrasonic scanning and monitoring functions according to claim 1, further comprising:
a scanning period setting unit (14) for setting the predetermined time interval and/or a duration of each scanning.

4. The monitor with ultrasonic scanning and monitoring functions according to claim 3, further comprising:
a storage unit (16) for storing the common physiological parameter detected by the monitoring function module and/or the processed echo signals obtained by the ultrasonic function module within a predetermined time.

5. The monitor with ultrasonic scanning and monitoring functions according to claim 3, wherein the process performed on the echo signals by the ultrasonic function module (12) comprises: obtaining a digital-processing-step signal, and calculating the digital-processing-step signal to obtain a numeric-type parameter, a waveform or a trend chart, wherein the digital-processing-step signal is one of a radio frequency signal, a baseband signal, and an envelope signal.

6. The monitor with ultrasonic scanning and monitoring functions according to claim 3, wherein the process performed on the echo signal by the ultrasonic function module (12) comprises: performing digital process on the echo signals to obtain a digital-processing-step signal, acquiring an ultrasonic image based on the digital-processing-step signal and a selected imaging mode, and calculating the ultrasonic image to obtain a numeric-type parameter, a waveform or a trend chart, wherein the imaging mode supported by the ultrasonic function module is at least one of B-mode imaging mode, M-mode imaging mode, color Doppler imaging mode, pulse wave imaging mode, elasticity imaging, 3D imaging mode and 4D imaging mode.

7. The monitor with ultrasonic scanning and monitoring functions according to claim 1, wherein the display apparatus comprises:
a common monitoring information display unit (30) for displaying the common physiological parameter monitored by the monitoring function module; and
an ultrasonic information display unit (32) for displaying the processed echo signals obtained by the ultrasonic function module.

8. The monitor with ultrasonic scanning and monitoring functions according to claim 1, wherein the ultrasonic sensor comprises a plurality of ultrasonic transducers each connected to the ultrasonic function module, each ultrasonic transducer being attached on the tagged object's different positions of body surface, wherein the plurality of ultrasonic transducers scan the tagged object synchronously and in real time.

9. The monitor with ultrasonic scanning and monitoring functions according to claim 1, wherein the common physiological parameter monitored by the monitoring function module comprises at least one of electrocardiogram monitoring parameter, blood pressure, oxygen saturation parameter and respiration parameter, and the ultrasonic function module obtains at least one of cardiac ejection fraction, left ventricular fractional shortening, stroke volume, cardiac output, cardiac index, left ventricular end-diastolic volume and left ventricular end-systolic volume after processing the echo signals of the tagged object.

10. The monitor with ultrasonic scanning and monitoring functions according to claim 8, wherein the monitoring function module and the ultrasonic function module are integrated in a host of the monitor.

11. The monitor with ultrasonic scanning and monitoring functions according to claim 8, wherein the monitoring function module and the ultrasonic function module are independent of each other.

12. The monitor with ultrasonic scanning and monitoring functions according to claim 11, wherein the monitoring function module and the ultrasonic function module are connected directly or connected via a host of the monitor.

13. The monitor with ultrasonic scanning and monitoring functions according to claim 8, wherein the monitoring function module and the ultrasonic function module are detachable modules independent of each other, each pluggable connecting with a host of the monitor; and the host provides a corresponding pluggable interface for each module.

14. A monitoring method for synchronously monitoring a common physiological parameter of a tagged object and ultrasonic scanning, the method comprising:
monitoring a common physiological parameter of a tagged object through the monitoring function module, and displaying the monitored common physiological parameter on the display apparatus; and
performing ultrasonic scanning on human tissue through the ultrasonic function module to obtain echo signals of the tagged object, processing the echo signals to form an image, a waveform or a numeric-type parameter and display on the display apparatus.

15. The monitoring method according to claim 14, wherein the step of performing ultrasonic scanning on human tissue through the ultrasonic function module to obtain echo signals of the tagged object comprises:
allowing at least one ultrasonic probe to be directly attached tightly on a correspondingly fixed position of the tagged object's body surface, scanning a predetermined slice plane at each corresponding position of the tagged object to obtain echo signals from the corresponding position.

16. The monitoring method according to claim 14, wherein the step of processing the echo signals to form an image, a waveform or a numeric-type parameter and display on the display apparatus comprises:
obtaining a digital-processing-step signal by performing digital process on the echo signals, and calculating the digital-processing-step signal according to the digital-processing-step signal to obtain a numeric-type parameter, a waveform or a trend chart.

17. The monitoring method according to claim 16, wherein the digital-processing-step signal is one of a radio frequency signal, a baseband signal, and an envelope signal.

18. The monitoring method according to claim 14, wherein the step of processing the echo signal to form an image, a waveform or a numeric-type parameter and display on the display apparatus comprises:
performing digital process on the echo signals to obtain a digital-processing-step signal, acquiring an ultrasonic image based on the digital-processing-step signal and a selected imaging mode, and calculating the ultrasonic image to obtain a numeric-type parameter, a waveform or a trend chart, wherein the imaging mode supported by the ultrasonic function module is at least one of B-mode imaging mode, M-mode imaging mode, color Doppler imaging mode, pulse wave imaging mode, elasticity imaging mode, 3D imaging mode and 4D imaging mode.

19. An ultrasonic apparatus for monitoring, comprising an ultrasonic function module (12), an ultrasonic sensor (22) and a display apparatus (3), wherein:
the ultrasonic sensor (22) is coupled to the ultrasonic function module and scans a predetermined slice plane of a tagged object to obtain echo signals of the tagged object;
the ultrasonic function module (12) monitors the tagged object by ultrasonically scanning the tagged object with a predetermined time interval to obtain and process the echo signals of the tagged object acquired from the ultrasonic sensor; and
the display apparatus (3) is coupled to the ultrasonic function module, receives and displays the processed echo signals derived from the ultrasonic function module.

20. The ultrasonic apparatus for monitoring according to claim 19, wherein the ultrasonic sensor is attached tightly on a fixed position of the tagged object's body surface, so as to scan the predetermined slice plane of the tagged object.

21. The ultrasonic apparatus for monitoring according to claim 20, wherein the ultrasonic function module further comprises:
a scanning period setting unit (14) for setting the predetermined time interval and/or a duration of each scanning.

22. The ultrasonic apparatus for monitoring according to claim 21, wherein the ultrasonic function module further comprises:
a storage unit (16) for storing the processed echo signals obtained by the ultrasonic function module within a predetermined time.

23. The ultrasonic apparatus for monitoring according to claim 22, wherein the process performed on the echo signals of the tagged object by the ultrasonic function module comprises: calculating the echo signals to obtain a numeric-type parameter, a waveform or a trend chart.

24. The ultrasonic apparatus for monitoring according to claim 22, wherein the process performed on the echo signals of the tagged object by the ultrasonic function module comprises: performing digital process on the echo signals to obtain a digital-processing-step signal, acquiring an ultrasonic image based on the digital-processing-step signal and a selected imaging mode, and calculating the ultrasonic image or the digital-processing-step signal to obtain a numeric-type parameter, a waveform or a trend chart, wherein the imaging mode supported by the ultrasonic function module is at least one of B-mode imaging mode, M-mode imaging mode, color Doppler imaging mode, pulse wave imaging mode, elasticity imaging mode, 3D imaging mode and 4D imaging mode.

25. The ultrasonic apparatus for monitoring according to claim 24, wherein the display apparatus is further provided with:
a monitoring information display unit (34) for displaying the numeric-type parameter, the waveform or the trend chart acquired by the ultrasonic function module; and
an ultrasonic image display unit (36) for displaying the ultrasonic image obtained by the selected imaging mode according to the processed echo signals performed by the ultrasonic function module.

26. The ultrasonic apparatus for monitoring according to claim 19, wherein the ultrasonic sensor is a plurality of ultrasonic transducers connected to the ultrasonic function module, each ultrasonic transducer being attached on the tagged object's different position of body surface, wherein the plurality of ultrasonic transducers scan the tagged object synchronously and in real time.

27. The ultrasonic apparatus for monitoring according to claim 26, wherein the ultrasonic function module obtains at least one of cardiac ejection fraction, left ventricular fractional shortening, stroke volume, cardiac output, cardiac index, left ventricular end-diastolic volume and left ventricular end-systolic volume after processing the echo signals of the tagged object.

28. The ultrasonic apparatus for monitoring according to claim 25, wherein the display apparatus is provided with a display screen having at least one window for displaying the numeric-type parameter, the waveform or the trend chart by default according to the monitoring information display unit; and the display screen is provided with a window which is triggered to display the ultrasonic image according to the monitoring information display unit.

29. The ultrasonic apparatus for monitoring according to claim 28, wherein the window displaying the ultrasonic image is independent of the window displaying the numeric-type parameter, the waveform or the trend chart.

30. The ultrasonic apparatus for monitoring according to claim 28, wherein the window displaying the ultrasonic image is or one of the window displaying the numeric-type parameter, the waveform or the trend chart.
